(19) United States
(12) Patent Application Publication
(10) Pub. No.: US 2011/0003694 A1
(43) Pub. Date: Jan. 6, 2011

(54) USE OF SOLUTIONS OF MINT OIL OR OF L-CARVONE FOR THE FUNGICIDAL AND/OR ANTI-GERMINATING TREATMENT OF BULBS AND/OR TUBERS

(75) Inventor: Alberto Sardo, Chateaurenard (FR)

Correspondence Address: SUGHRUE MION, PLLC, 2100 PENNSYLVANIA AVENUE, N.W., SUITE 800, WASHINGTON, DC, 20037, US

(73) Assignee: Zone Artisanale La Crau Route Nationale 7

(21) Appl. No.: 12/741,934

(22) PCT Filed: Nov. 10, 2008
(86) PCT No.: PCT/FR08/52022

§ 371 Date: Sep. 14, 2010

(30) Foreign Application Priority Data
Nov. 9, 2007 (FR) 07 58919

(51) Int. Cl. A01N 65/22; A01P 3/00; A01P 13/00; A01H 3/04; A01N 35/00

(52) U.S. Cl. 504/118; 424/747; 504/182; 504/174; 514/690

(57) ABSTRACT

The present invention relates to a novel method of treating bulbs or tubers with L-carvone and/or mint oil by thermal fogging.

The present invention relates to a method for treating bulbs or tubers, in particular an anti-germinating and fungicidal treatment. In particular, said method makes it possible to decontaminate or inhibit fungal contamination.

Field crops are subject to fungal infection in the field or during storage following harvesting. This applies in particular to potatoes, which may be infected by the Rhizoctonia solani fungus. This causes black scurf and develops from black sclerotia attached to the parent tuber or present in the soil. Tubers from affected plants are small, misshapen and angular. Contamination by Rhizoctonia solani usually occurs in the field and develops during storage. It is therefore important to use a fungicidal treatment in the field and after harvesting.

The potato is also subject to silver scurf (Helminthosporium solani).

Moreover, during storage bulbs and tubers are subject to germination which should be avoided or limited in order to commercialise said bulbs or tubers.

It is therefore useful to have novel and/or improved fungicidal treatments available, particularly of natural origin, presenting no danger for consumers.

The use of L-carvone for the anti-germinating treatment of potatoes has been described (see in particular application EP 0 719 499). However, this method consists of the application once a month of doses of between 5 and 50 g of L-carvone per tonne of tubers by thermal fogging. This document does not disclose any fungicidal activity; moreover, the application pattern does not produce a satisfactory fungicidal effect. Further, the fungicidal activity of L-carvone in vitro has also been described by Goris et al., Brighton Crop Protection Conference, Pest and Disease, 1994, 307-312). However, this article only describes experimental tests on a laboratory scale which are not necessarily indicative of the industrial scale. Moreover, the essential mint oil is used by evaporation and the article does not suggest any doses and/or application frequency.

It is therefore advantageous to make available a new crop treatment method using mint oil or L-carvone to obtain the anti-germinating and fungicidal effect.

According to the first object, the present invention therefore relates to a fungicidal and/or anti-germinating crop treatment method with L-carvone and/or mint oil by thermal fogging, comprising an initial application of 60 to 400 g of L-carvone and/or mint oil per tonne, then one or more applications at a frequency of between three days and six weeks.

Preferably, the fungicidal and anti-germinating activities are obtained together at doses and frequencies according to the invention.

According to a preferred aspect, the method according to the invention aims to decontaminate or prevent contamination by Rhizoctonia such as Rhizoctonia solani, scurf such as silver scurf, in particular Helminthosporium solani, or mildew such as Phytophthora infestans. The method according to the invention is particularly advantageous for the fungicidal and anti-germinating treatment of the potato.

According to another preferred aspect, the doses for the first application are between 60 and 200 g of L-carvone and/or mint oil per tonne of bulbs or tubers. The subsequent application or applications may be carried out at doses of between 20 and 100 g per tonne.

Preferably, the frequency of the subsequent application or applications is between three days and four weeks, more preferably between three days and three weeks.

In general, said composition is advantageously applied during storage to harvested bulbs or tubers.

According to a particular embodiment, the composition may be diluted, in particular in an organic solvent.

Dipropylene glycol, monopropylene glycol, diisobutyl ketone, ethyl alcohol, isopropyl alcohol or mixtures thereof may be cited as organic solvents.

Carvone refers to 2-methyl-5-(1-methylethenyl)-2-cyclohexen-1-one. Unlike D-carvone, of which the principal natural source is cumin, and other oxygenated monoterpenes, L-carvone is a compound which is naturally ingested in large quantities in human nutrition, for example when consuming some mint-based dishes or when chewing gum, its main natural source being spearmint or Mentha spicata. L-carvone may be obtained from natural sources, in particular spearmint (Mentha spicata) or by synthesis from limonene. The development of an effective fungicidal treatment using a product widely consumed in nutrition as the active ingredient meets current needs for non-toxic treatments.

The expression “anti-germinating treatment” refers to the prevention and/or inhibition of germination in bulbs and tubers.

The expression “fungicidal treatment” refers to decontamination from or prevention of contamination by fungus, particularly scurf, rhizoctonia or mildew. Silver scurf (Helminthosporium solani), Rhizoctonia solani and Phytophthora infestans affect potatoes in particular.

Compositions are prepared by mixing the components according to a method known per se.

The following are given as non-limiting illustrative examples of the present invention.

Thermal fogging is a process consisting of the application to fruit and vegetables in particular of an extremely fine mist, the droplets of which have a size of approximately one micrometre, produced by injecting a liquid into a current of hot air which serves as a carrier for chemical substances which form the active ingredients, the mist thus produced ensuring coating by the treatment product. The thermal fogging process is described in particular in EP 0 719 499. It may advantageously be implemented using the Electrofog® equipment commercialised by Xeda.

The compositions are prepared by mixing the components by a method known per se.

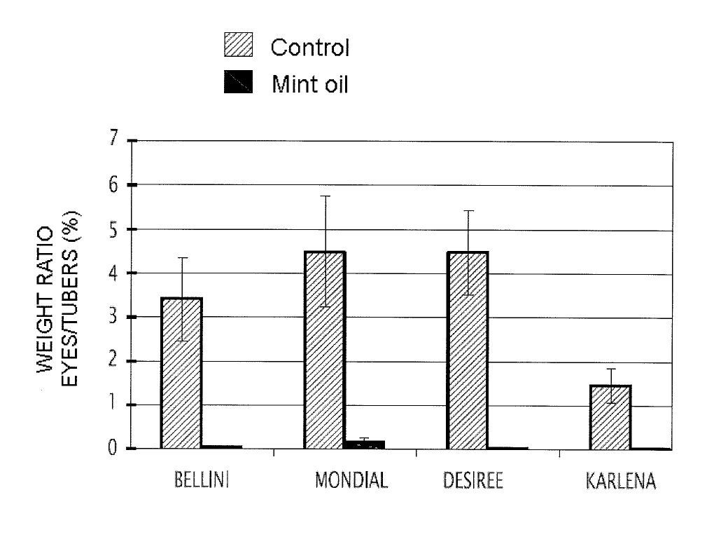

FIG. 1 illustrates the thermal fogging effect of mint oil on the germination of stored potatoes. The tubers of four varieties were stored for six months at 8° C. and 95% humidity and thermally fogged monthly with a 100 ml/t dose of mint oil for the first application and 30 ml/t for each subsequent application. The error bars represent the standard deviation.

The following are given as non-limiting illustrative examples of the present invention.

### EXAMPLES

### Example 1

Potatoes (Désirée cultivar) were stored at 8° C. after harvesting. Three weeks after harvesting, they were treated with natural mint oil containing 75-80% L-carvone. The first treatment (90 ml/tonne of potatoes) was followed 21 days later by the second treatment (30 ml/tonne of potatoes), and subsequent treatments were carried out at 45-day intervals (30 ml/tonne of potatoes). All the treatments were carried out using the Xeda Electrofog thermal fogger.

After four months of storage at 8° C., the potatoes were transferred to 20° C. for planting. Two to three weeks later, the potatoes began to germinate. The germinated potatoes were planted in the fields. The development of treated and untreated potatoes was identical. The harvest from plants treated with mint oil was not infected by Rhizoctonia solani whereas the potatoes from untreated batches were infected. These results demonstrate that mint oil can be used effectively to limit the contamination of potatoes by Rhizoctonia solani.

### Example 2

### L-carvone activity on silver scurf in potatoes

Roseval potatoes were treated by thermal fogging with mint oil at 90 g/tonne, or with 67-72 g/tonne of L-carvone, then three times at 30 g/tonne, or 22.5-24 g/tonne of L-carvone, at 21-day intervals. They were then stored for three months at 8° C. The silver scurf coverage rates were measured on the batches treated with L-carvone and on the controls treated by thermal fogging with CIPC at 200 g/L at a dose of 60 ml/tonne for the first application then at 40 ml/tonne for the subsequent applications. The results are shown in the table below:

TABLE 1
Effectiveness of silver scurf on Roseval after three months storage

| | | Coverage | Coverage | Coverage | Coverage | Coverage | |
|---|---|---|---|---|---|---|---|
| | Undamaged | ≦5% | 6-25% | 26-50% | 5-75% | >75% | Silver scurf |
| | tubers (%) | (%) | (%) | (%) | (%) | (%) | index |
| | Class 0 | Class 1 | Class 2 | Class 3 | Class 4 | Class 5 | (0-100) |
| L-carvone | 71.5 a | 28.5 | 0.0 b | 0.0 b | 0.0 | 0.0 | 0.8 b |
| Thermo | 60.2 b | 26.2 | 7.7 a | 3.9 a | 0.7 | 1.5 | 5.7 a |
| reference | | | | | | | |
| C.V. | 3.55% | 8.54% | 34.66% | 70.53% | 157.77% | 145.57% | 32.40% |
| Significance | H.S. | N.S. | H.S. | H.S. | N.S. | N.S. | H.S. |

### Example 3

The following four varieties of potatoes were used: Bellini, Mondial, (Désirée and Karlena. Mint oil commercialised by Xeda International, Saint-Andiol, France, was applied by thermal fogging (Electrofog, Xeda International).

The following treatments were compared: no treatment, dose of 100 ml/tonne for the first application and 30 ml/tonne monthly for each subsequent application.

Monthly thermal fogging began after two weeks of storage during which the temperature was gradually reduced to 8° C. During application, air inlets were closed and the humidifier turned off. The tubers were stored in plastic boxes each weighing 100 kg. The treatment was applied in storage chambers measuring 8 m³, at 8° C. and 95% humidity.

Thermal fogging of the tubers with mint oil led to highly effective penetration in the stock of tubers and to inhibition of the germination process. The four varieties were stored for over nine months without germination. The untreated tubers began to germinate after three months of storage and the eyes were 4% of the weight of the tuber.

The results are shown in FIG. 1.

### Example 4

Applications of Xeda HM Nat (55% L-carvone) were carried out by thermal fogging at 180° C. on Bintje potatoes stored in a cell, using the Xeda Electrofog.

These cells, with a capacity of about 12 tonnes, are specially designed for this type of testing, and are isolated, ventilated and equipped with a homogenous air distribution system (letter box) in potato storage bins (18 bins per cell).

The cells are maintained at the required temperature by the introduction of cool air from the outside applying the principle of air mixing used in practice in agricultural enterprises with the additional support, if required, of a cooling unit positioned inside the cell. The required temperature during storage is 7.5° C.

The first application is made at a dose of 80 g/t of commercial product and subsequent applications are made every three weeks at a dose of 30 g/t. For each grading, samples in mesh bags are placed in the centre of the bins among the fill-in tubers for removal on the required dates to be analysed. All the bins with samples are distributed evenly in the storage building.

The L-carvone residue in the treated potatoes was analysed in the following manner:

Steam distillation: hydrodistillation

Recovery in hexane

Analysis in gas phase chromatography coupled to a mass spectrometer

Limit of determination: 0.02 mg/kg

Recovery rate: 114% at 0.05 mg/kg
92% at 0.13 mg/kg

Residue level in the specimens:

| | Laboratory | L-carvone residue |
|---|---|---|
| Code | references | (mg/kg) |
| BI LC | PC 97 pe | 0.02-0.12 |
| Control potatoes | PC 97 PE 570 | <0.02 |

The concentration of residue obtained may be considered negligible.

This lack of residue therefore ensures conformity with regulations and safety for consumers.

1. Method for the fungicidal and/or anti-germinating treatment of bulbs or tubers, comprising the application by thermal fogging of mint oil, L-carvone or mixtures thereof, optionally diluted in an organic solvent, at an initial dose of L-carvone of between 60 g and 400 g per tonne of bulbs or tubers, then one or more applications repeated at a frequency of between three days and six weeks.

2. Method according to claim 1, characterised in that the initial dose of L-carvone is between 60 g and 200 g per tonne of bulbs or tubers.

3. Method according to claim 1, characterised in that the frequency of the subsequent application or applications is between three days and three weeks.

4. Method according to claim 1, characterised in that said diluted composition comprises at least 30% by weight of mint oil and/or L-carvone.

5. Method according to claim 1, characterised in that said organic solvent is selected from monopropylene glycol, diisobutyl ketone, ethyl alcohol, isopropyl alcohol or mixtures thereof.

6. Method according to claim 1, characterised in that the subsequent doses are between 20 g and 100 g of active ingredient per tonne.

7. Method according to claim 1, characterised in that said method is implemented in storage chambers.

8. Method according to any claim 1, characterised in that its purpose is decontamination from or prevention of contamination by one or more fungi selected from Rhizoctonia solani, Helminthosporium solani and Phytophthora infestans.

9. Method according to claim 1, characterised in that potatoes are treated.
